(19) United States
(12) Patent Application Publication
(10) Pub. No.: US 2014/0308230 A1
(43) Pub. Date: Oct. 16, 2014

(54) PRODUCT FOR TEMPORARILY SHAPING KERATIN FIBERS ON THE BASIS OF A COMBINATION OF SPECIFIC FILM-FORMING POLYMERS

(71) Applicant: Henkel AG & Co. KGaA, Dusseldorf (DE)

(72) Inventors: Diane Metten, Hamburg (DE); Bernd Richters, Hamburg (DE); Rene Scheffler, Ellerau (DE)

(73) Assignee: Henkel AG & Co. KGaA, Dusseldorf (DE)

(21) Appl. No.: 14/367,832

(22) PCT Filed: Nov. 9, 2012
(86) PCT No.: PCT/EP2012/072278

§ 371 Date: Jun. 20, 2014

(30) Foreign Application Priority Data
Dec. 20, 2011 (DE) 10 2011 089 169.2

(51) Int. Cl. A61K 8/81; A61K 8/41; A61Q 5/06; A61K 8/898

(52) U.S. Cl. CPC A61K 8/8182; A61K 8/8117; A61K 8/898; A61K 8/416; A61Q 5/06; A61K 8/817
USPC 424/70.122; 424/70.11; 424/70.15

(57) ABSTRACT

A cosmetic agent for temporarily deforming keratinic fibers is provided. In an exemplary embodiment, a cosmetic agent, contains in a cosmetically acceptable carrier a) at least one polystyrenesulfonate A), b) at least one copolymer B formed from at least one monomer (B1) selected from N-vinyllactams, at least one monomer (B2) selected from quaternized N-vinylimidazoles.

### CROSS REFERENCE TO RELATED APPLICATIONS

The present disclosure is a U.S. National Stage entry under 35 U.S.C. §371 based on International Application No. PCT/EP2012/072278, filed Nov. 9, 2012 which was published under PCT Article 21(2) and which claims priority to German Patent Application No. DE 10 2011 089 169.2 filed on Dec. 20, 2011, which is incorporated herein by reference in its entirety.

### TECHNICAL FIELD

The technical field describes cosmetic agents based on a specific polymer combination, use of these cosmetic agents for temporarily deforming keratinic fibers and cosmetic methods using these agents.

### BACKGROUND

Polymers are widely used in the most varied cosmetic agents. They are to be found in agents for treating skin as well as in agents for treating hair, in agents which are washed off or out again directly after use, i.e. “rinse-off products”, and in agents which remain on the skin or hair, i.e. “leave-on agents”. The polymers are used for the most varied reasons and specific properties of the polymers are exploited in each case. In agents for treating skin, in shampoos, hair rinses and hair masks, the emphasis often lies on the thickening or conditioning properties of the polymers. In agents for temporarily deforming keratinic fibers, hereinafter also known as styling agents, alongside these properties film-forming and/or setting effects are particularly desired. Polymers often also serve as auxiliaries for improving or indeed enabling deposition and fixing of other active substances and ingredients on the skin or hair. By adding suitable polymers to hair coloring agents, for example, rubbing fastness and coloring durability may be increased.

Cosmetic agents generally contain individual polymers which are specifically tailored to achieving a very specific effect. If various effects are to be achieved, a plurality of polymers must be added. However, using too many different polymers may be associated with a series of disadvantages. Problems may accordingly arise during formulation, for instance because the polymers react with one another or with other components of the agent resulting in precipitation or decomposition phenomena. Certain polymers also have a tendency to be deposited so permanently on the skin and in particular on the hair that they are no longer completely removed with normal washing and the polymer accumulates undesirably so ultimately leading to contamination of the skin or hair.

There is therefore a constant need for polymers or suitable combinations of small numbers of polymers which simultaneously exhibit as many as possible of the desired properties.

For example, in the case of styling agents, the polymers used need to give the treated hair the strongest possible hold. In addition to a high degree of hold, styling agents must meet a whole series of further requirements. These may be broadly divided into properties on the hair, properties of the respective formulation, for example properties of the foam, the gel or the sprayed aerosol, and properties which affect the handling of the styling agent, wherein properties on the hair are of particular importance. Particular mention should be made of moisture resistance, low tackiness and a well-balanced conditioning effect. Moreover, a styling agent should as far as possible be universally applicable for all hair types. If the styling agent is a gel or a paste, the polymers should additionally have thickening properties.

The disclosure provides further suitable polymer combinations which are distinguished by good film-forming and/or setting properties, have a very high level of hold without having to sacrifice flexibility and good moisture resistance, in particular perspiration and water resistance, and are additionally suitable for producing stably viscous and stably transparent cosmetic compositions.

### SUMMARY

These objects were achieved by a cosmetic agent having a specific polymer combination. An exemplary embodiment provides a cosmetic agent, containing in a cosmetically acceptable carrier

a) at least one polystyrenesulfonate A
b) at least one copolymer B formed from
at least one monomer (B1) selected from quaternized N-vinylimidazoles
at least one monomer (B2) selected from N-vinyllactams.
   In another embodiment, a method for temporarily deforming keratinic fibers is provided. The method includes applying onto the keratinic fibers a cosmetic agent, containing in a cosmetically acceptable carrier

a) at least one polystyrenesulfonate A

b) at least one copolymer B formed from
at least one monomer (B1) selected from quaternized N-vinylimidazoles, and
at least one monomer (B2) selected from N-vinyl lactams.

### DETAILED DESCRIPTION

Exemplary agents contain the active substances in a cosmetic carrier. In an exemplary embodiment, the cosmetic carrier is aqueous, alcoholic or aqueous-alcoholic. Exemplary aqueous-alcoholic carriers may be hydrous compositions containing from about 3 to about 70 wt. % of a C₁-C₄ alcohol, such as ethanol or isopropanol, relative to the total weight of the mixture for use. An exemplary aqueous carrier contains at least about 30 wt. %, such as at least a bout 50 wt. % water, relative to the total weight of the mixture for use.

Exemplary cosmetic agents contain, relative to the total weight thereof, from about 40 to about 99 wt. %, such as from about 50 to about 98 wt. %, for example from about 60 to about 95 wt. % or from about 70 to about 90 wt. % water. The pH value (10% solution, 20° C.) of exemplary cosmetic agents amounts to from about 4 to about 9, such as from about 5 to about 8, for example from about 6 to about 7.

Exemplary cosmetic agents contain as first essential component at least one polystyrenesulfonate A. Exemplary polystyrenesulfonates are distinguished by an elevated molecular weight. An exemplary polystyrenesulfonate A has a molecular weight of from about 80 to about 180 kDa, such as from about 100 to about 160 kDa, for example from about 120 to about 140 kDa.

The proportion by weight of copolymer A in the total weight of exemplary cosmetic agents amounts to from about 0.05 to about 10 wt. %, such as from about 0.1 to about 7.0 wt. %, for example from about 0.2 to about 5.0 wt. %.

Exemplary agents contain as second essential component at least one copolymer B formed from

at least one monomer (B 1) selected from quaternized N-vinylimidazoles,

at least one monomer (B2) selected from N-vinyllactams,

Exemplary agents are characterized in that they contain at least one copolymer B which
contains at least one structural unit according to formula (B4), in which
   R means a C₁ to C₃₀ alkyl group, a C₁ to C₄ aralkyl group, a C₂ to C₆ alkenyl group or a C₂ to C₆ hydroxyalkyl group and
   X⁻ denotes a physiologically acceptable anion and
contains at least one further structural unit according to formula (B-II), in which n denotes 1, 2 or 3 as the number of methyl units.

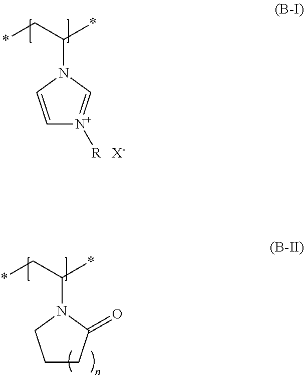

Film-forming and/or setting copolymers B are known. These copolymers have at least one structural unit according to formula (B-I) and at least one structural unit according to formula (B-II) and may additionally comprise further structural units, which are incorporated by polymerization through the addition of corresponding monomers during polymerization.

In formula (B-I), R denotes a C₁ to C₃₀ alkyl group, a C₁ to C₄ aralkyl group, a C₂ to C₆ alkenyl group or a C₂ to C₆ hydroxyalkyl group. Examples of exemplary groups R are —CH₃; —CH₂CH₃, —CH₂CH₂CH₃, CH(CH₃)₂, —(CH₂)₃CH₃, —CH₂—CH(CH₃)₂, CH(CH₃)CH₂CH₃, —C(CH₃)₃, —CH₂OH, —CH₂CH₂OH, —CH₂CH₂CH₂OH, —CH(OH)CH₂CH₃, —CH₂CH(OH)CH₃. X⁻ denotes a physiologically acceptable anion, exemplary anions being chloride, bromide, iodide, sulfate, methosulfate, ethyl sulfate, tosylate and tetrafluoroborate. Exemplary agents are characterized in that the monomer (B1) is a salt of 3-alkyl-1-vinylimidazolium with physiologically acceptable anions, such as 3-methyl-1-vinylimidazolium methylsulfate.

In formula (B-II), n denotes the number of methylene groups. With n=1 formula (B-II) denotes a vinylpyrrolidone unit, with n=2 it denotes a vinylpiperidinone unit and with n=3 it denotes a vinylcaprolactam unit. Exemplary agents are characterized in that monomer (B2) is selected from N-vinylcaprolactam and N-vinylpyrrolidone. In an exemplary embodiment, N-Vinylpyrrolidone is used as monomer (B2).

Exemplary agents are characterized in that they contain as copolymer B a copolymer B1 which
contains at least one structural unit according to formula (B-I), in which R denotes a methyl group and X denotes methosulfate,
contains at least further structural units according to formula (B-II), in which n denotes 1 methylene unit.

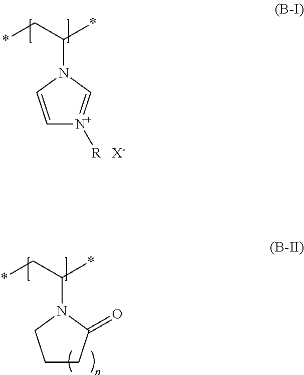

Exemplary copolymers B1 contain from about 10 to about 30 mol %, such as from about 15 to about 25 mol %, for example about 20 mol % structural units according to formula (B-I), and from about 70 to about 90 mol %, such as from about 75 to about 85 mol %, for example about 80 mol % structural units according to formula (B-II).

In exemplary embodiments, the copolymers B1 contain, in addition to polymer units which arise from the incorporation of the stated structural units according to formula (B-I) and (B-II) into the copolymer, at most about 5 wt. %, such as at most about 1 wt. %, of polymer units originating from the incorporation of other monomers. Exemplary copolymers B1 are exclusively synthesized from structural units of formulae (B-I) and (B-II) and may be described by the general formula

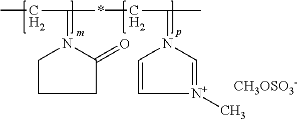

wherein the indices m and n vary depending on the molar mass of the polymer and are not intended to mean that the copolymers are block copolymers. Instead, structural units of the formula (B-I) and the formula (B-II) may be present randomly distributed in the molecule. Such N-methylvinylimidazole/vinylpyrrolidone copolymers are designated POLYQUATERNIUM-44 according to INCI nomenclature and are obtainable for example from BASF under the trade name Luviquat® UltraCare.

Exemplary agents contain a copolymer B1 which has molar masses within a specific range. Exemplary hair deforming agents include the copolymer B1 having a molar mass of from about 50 to about 400 kDa, such as from about 100 to about 300 kDa, for example from about 150 to about 250 kDa or from about 190 to about 210 kDa.

In addition to the copolymer(s) B1 or instead of the copolymer(s) B1, the exemplary agents may also contain copolymers B2 which comprise as additional structural units structural units of formula (B-II), in which n denotes the number 3.

Exemplary agents are characterized in that they contain as copolymer B a copolymer B2 which
contains at least one structural unit according to formula (B-I), in which R denotes a methyl group and X denotes methosulfate,
contains at least further structural units according to formula (B-II), in which n denotes 1 methylene unit
contains at least further structural units according to formula (B-II), in which n denotes 3 methylene units.

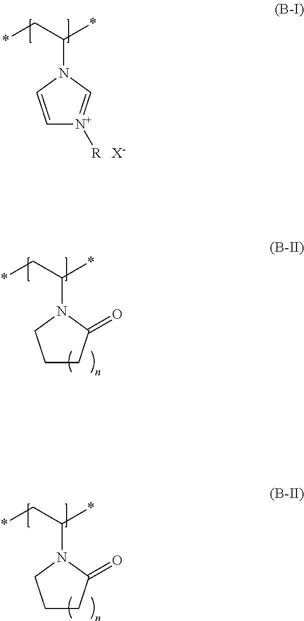

Exemplary copolymers B2 contain, in addition to polymer units which arise from the incorporation of the stated structural units according to formula (B-I) and (B-II) into the copolymer, at most about 5 wt. %, such as at most about 1 wt. %, of polymer units originating from the incorporation of other monomers. Exemplary copolymers B2 are exclusively synthesized from structural units of the formulae (B-I) and (B-II) and may be described by the general formula

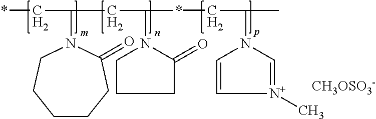

wherein the indices m, n and p vary depending on the molar mass of the polymer and are not intended to mean that the copolymers are block copolymers. Instead, structural units of the formula (B-I) and the formula (B-II) may be present randomly distributed in the molecule.

Such N-methylvinylimidazole/vinylpyrrolidone/vinylcaprolactam copolymers are designated POLYQUATERNIUM-46 according to INCI nomenclature and are obtainable for example from BASF under the trade name Luviquat® Hold.

Exemplary copolymers B2 contain from about 1 to about 20 mol %, such as from about 5 to about 15 mol %, for example about 10 mol % of structural units according to formula (B-I), and from about 30 to about 50 mol %, such as from about 35 to about 45 mol %, for example about 40 mol % of structural units according to formula (B-II) with n=1, and from about 40 to about 60 mol %, such as from about 45 to about 55 mol %, for example about 60 mol % of structural units according to formula (B-II) with n=3.

Exemplary agents contain a copolymer B2 which has molar masses within a specific range. Exemplary hair deforming agents include copolymer B2 having a molar mass of from about 100 to about 1000 kDa, such as from about 250 to about 900 kDa, for example from about 500 to about 850 kDa or from about 650 to about 710 kDa.

In addition to copolymer(s) B1 and/or B2 or instead thereof, the agents may also contain copolymers B3, which comprise, as additional structural units, structural units of formula (B-II), in which n denotes the number 3, further structural units from the group of vinylimidazole units, and further structural units from the group of acrylamide and/or methacrylamide units.

Exemplary agents are characterized in that copolymer B is formed from
at least one monomer (B 1) selected from quaternized N-vinylimidazoles,
at least one monomer (B2) selected from N-vinyllactams,
the monomer N-vinylimidazole (B3) and
at least one monomer (B4) selected from acrylic acid amide, methacrylic acid amide, N-alkyl acrylic acid amide and N-alkyl methacrylic acid amide.

Exemplary agents are characterized in that they contain as copolymer B a copolymer B3 which
contains at least one structural unit according to formula (B-I), in which R denotes a methyl group and X denotes methosulfate,
contains at least further structural units according to formula (B-II), in which n denotes 1 methylene unit
contains at least further structural units according to formula (B-III)
contains at least further structural units according to formula (B-IV)

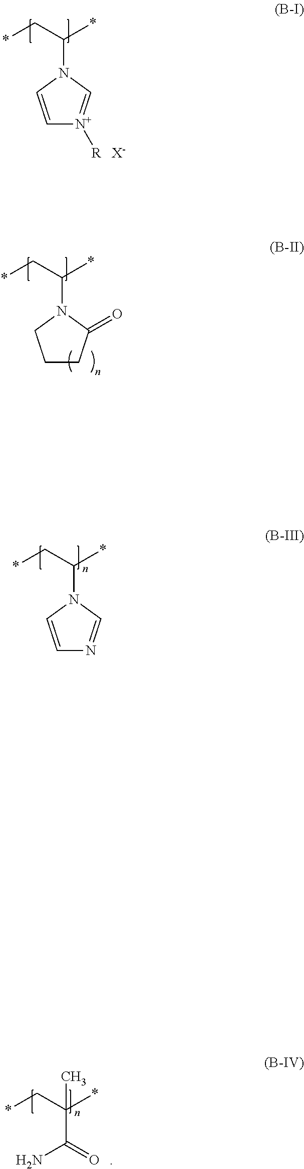

Exemplary copolymers B3 contain, in addition to polymer units which arise from the incorporation of the stated structural units according to formula (B-I), (B-II), (B-III) and (B-IV) into the copolymer, at most about 5 wt. %, such as at most about 1 wt. %, of polymer units originating from the incorporation of other monomers. Exemplary copolymers B3 are exclusively synthesized from structural units of formulae (B-I), (B-II), (B-III) and (B-IV) and may be described by the general formula

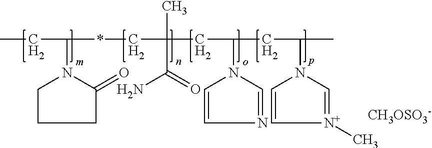

wherein the indices m, n, o and p vary depending on the molar mass of the polymer and are not intended to mean that the copolymers are block copolymers. Instead structural units of formulae (B-I), (B-II), (B-III) and (B-IV) may be present randomly distributed in the molecule.

Exemplary agents are characterized in that copolymer B is formed from
3-methyl-1-vinylimidazolium methylsulfate (B1),
at least one monomer (B2) selected from N-vinylcaprolactam and N-vinylpyrrolidone,
N-vinylimidazole (B3) and
at least one monomer (B4) selected from acrylic acid amide and methacrylic acid amide.

Exemplary agents are those in which copolymer B is formed from
3-methyl-1-vinylimidazolium methylsulfate (B1),
N-vinylpyrrolidone (B2),
N-vinylimidazole (B3) and
methacrylic acid amide (B4).

Exemplary copolymer B includes in a proportion of at least about 70 wt. %, such as at least about 80 wt. %, for example at least about 90 wt. % or at least about 95 wt. % of the structural units (B1), (B2), (B3) and (B4).

Such N-methylvinylimidazole/vinylpyrrolidone/vinylcaprolactam copolymers are designated POLYQUATERNIUM-68 according to INCI nomenclature and are obtainable for example from BASF under the trade name Luviquat® Supreme.

Exemplary copolymers B3 contain from about 1 to about 12 mol %, such as from about 3 to about 9 mol %, for example about 6 mol % of structural units according to formula (B-I), and from about 45 to about 65 mol %, such as from about 50 to about 60 mol %, for example about 55 mol % of structural units according to formula (B-II) with n=1, and from about 1 to about 20 mol %, such as from about 5 to about 15 mol %, for example about 10 mol % of structural units according to formula (B-III), and from about 20 to about 40 mol %, such as from about 25 to about 35 mol %, for example about 29 mol % of structural units according to formula (B-IV).

Exemplary agents contain a copolymer B3 which has molar masses within a specific range. Exemplary hair deforming agents include copolymer B3 having a molar mass of from about 100 to about 500 kDa, such as from about 150 to about 400 kDa, for example from about 250 to about 350 kDa or from about 290 to about 310 kDa.

The proportion by weight of copolymer B in the total weight of exemplary cosmetic agents amounts to from about 0.05 to about 10 wt. %, such as from about 0.1 to about 7.0 wt. %, for example from about 0.2 to about 5.0 wt. %.

Exemplary agents are distinguished from cosmetic agents with alternative vinylpyrrolidone copolymers not only by the above-stated advantages but in particular also by an improved level of hold. For the cosmetic properties of exemplary agents, a weight ratio of polymers A and B is provided in the cosmetic agent of from about 8:1 to about 1:8, such as from about 6:1 to about 1:6, for example from about 4:1 to about 1:4.

Copolymer A is used in the exemplary cosmetic agents in partially neutralized or neutralized form. In an exemplary embodiment, at least one alkanolamine is used for neutralization. Exemplary alkanolamines usable as an alkalizing agent are selected from primary amines with a C₂-C₆ alkyl parent substance which bears at least one hydroxyl group. Exemplary alkanolamines are selected from the group formed from 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Exemplary alkanolamines are selected from the group 2-aminoethan-1-ol, 2-amino-2-methylpropan-1-ol and 2-amino-2-methylpropane-1,3-diol. 2-Amino-2-methylpropanol is a suitable neutralizing agent. Exemplary cosmetic agents contain at least one alkanolamine, such as 2-amino-2-methylpropanol. 2-Amino-2-methylpropanol is used in exemplary agents in a quantity which does not exceed the quantity needed to neutralize copolymer A. The quantities of 2-amino-2-methylpropanol used in exemplary agents amounts to from about 80 to about 100%, such as from about 90 to about 100%, for example from about 95 to about 100% of the quantity required for complete neutralization of copolymer A. In an exemplary embodiment, the proportion by weight of 2-amino-2-methylpropanol in the total weight of the cosmetic agent amounts to from about 0.1 to about 4.0 wt. %, such as from about 0.2 to about 3.0 wt. %, for example from about 0.5 to about 2.0 wt. %.

In addition to the previously described copolymers and carrier substances, exemplary cosmetic agents may contain further ingredients. The group of these further ingredients in particular includes cosmetically active auxiliary substances and additives.

Exemplary cosmetic agents contain at least one quaternary ammonium compound. Monomeric or polymeric active substances may be used as the quaternary ammonium compound.

From the plurality of possible monomeric quaternary ammonium compounds, the compounds from the groups:

trimethylalkylammonium halides;

esterquats

quaternary imidazolines

are effective.

The group of trimethylalkylammonium halides in particular includes the compounds of formula (Tkat1).

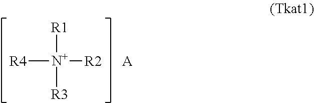

In the formula (Tkat1), R1, R2, R3 and R4 in each case mutually independently denote hydrogen, a methyl group, a phenyl group, a benzyl group, a saturated, branched or unbranched alkyl residue with a chain length of 8 to 30 carbon atoms, which may optionally be substituted with one or more hydroxyl groups. A denotes a physiologically acceptable anion, for example halides such as chloride or bromide and methosulfates.

Examples of compounds of formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide, behenyltrimethylammonium methosulfate. Exemplary cosmetic agents contain a monomeric quaternary ammonium compound from the group of trimethylalkylammonium halides.

Exemplary quaternary ammonium compounds are the cationic betaine esters of formula (Tkat1-2.1).

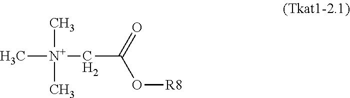

Exemplary esterquats are those with the trade names Armocare VGH-70, and Dehyquart® F-75, Dehyquart® L80, Stepantex® VS 90 and Akypoquat® 131.

A further group are quaternary imidazoline compounds. The formula (Tkat2) illustrated below shows the structure of these compounds.

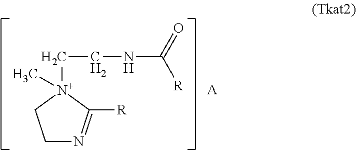

The residues R mutually independently in each case denote a saturated or unsaturated, linear or branched hydrocarbon residue with a chain length of 8 to 30 carbon atoms. Exemplary compounds of the formula (Tkat2) in each case contain the identical hydrocarbon residue for R. The chain length of exemplary residues R amounts to 12 to 21 carbon atoms. A denotes an anion as previously described. Examples are obtainable for example under INCI names Quaternium-27, Quaternium-72, Quaternium-83, Quaternium-87 and Quaternium-91. In an exemplary embodiment, Quaternium-91 is an exemplary residue.

With regard to cosmetic action, exemplary cosmetic agents have a proportion by weight of the monomeric quaternary ammonium compound in the total weight of the agent amounts to from about 0.05 to about 3.0 wt. %, such as from about 0.1 to about 2.0 wt. %, for example from about 0.2 to about 1.0 wt. %.

Suitable auxiliary substances and additives which may be mentioned are in particular additional conditioning substances.

An exemplary agent may contain at least one protein hydrolysate and/or one of the derivatives thereof as a conditioning substance of another compound class. Protein hydrolysates are product mixtures which are obtained by acidically, basically or enzymatically catalyzed degradation of proteins. As used herein, the term protein hydrolysates also covers total hydrolysates and individual amino acids and the derivatives thereof and mixtures of different amino acids. The molecular weight of exemplary protein hydrolysates which may be used is from about 75, the molecular weight of glycine, to about 200,000, such as from about 75 to about 50,000, for example from about 75 to about 20,000 daltons.

An exemplary agent may furthermore contain at least one vitamin, a provitamin, a vitamin precursor and/or one of the derivatives thereof as a conditioning substance.

Exemplary vitamins, provitamins and vitamin precursors are those which are conventionally assigned to groups A, B, C, E, F and H.

Like the addition of glycerol and/or propylene glycol, the addition of panthenol may increase the flexibility of the polymer film formed using the exemplary agent.

Exemplary agents may furthermore contain at least one plant extract, but also mono- or oligosaccharides and/or lipids as conditioning substance.

Oil bodies are furthermore suitable as a conditioning substance. Natural and synthetic cosmetic oil bodies include, for example, vegetable oils, liquid paraffin oils, isoparaffin oils and synthetic hydrocarbons and di-n-alkyl ethers having a total of between 12 and 36 C atoms, in particular 12 to 24 C atoms. Exemplary cosmetic agents contain at least one oil body, such as at least one oil body from the group of silicone oils. The group of silicone oils in particular includes dimethicones, which also include cyclomethicones, amino-functional silicones and dimethiconols. The dimethicones may be both linear and branched and cyclic or cyclic and branched. Suitable silicone oils or silicone gums are in particular dialkyl- and alkylarylsiloxanes, such as for example dimethylpolysiloxane and methylphenylpolysiloxane, and the alkoxylated, quaternized or also anionic derivatives thereof. Exemplary silicone oils or silicone gums are cyclic and linear polydialkylsiloxanes, the alkoxylated and/or aminated derivatives thereof, dihydroxypolydimethylsiloxanes and polyphenylalkylsiloxanes.

Ester oils, i.e. esters of C₆-C₃₀ fatty acids with C₂-C₃₀ fatty alcohols, such as monoesters of fatty acids with alcohols having 2 to 24 C atoms such as for example isopropyl myristate (Rilanit® IPM), isononanoic acid C₁₆₋₁₈ alkyl esters (Cetiol® SN), 2-ethylhexyl palmitate (Cegesoft® 24), stearic acid 2-ethylhexyl ester (Cetiol® 868), cetyl oleate, glycerol tricaprylate, coco fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are further exemplary conditioning oil bodies.

Additional suitable conditioning substances are dicarboxylic acid esters, symmetrical, asymmetrical or cyclic esters of carbonic acid with fatty alcohols, trifatty acid esters of saturated and/or unsaturated linear and/or branched fatty acids with glycerol, or fatty acid partial glycerides, which should be understood to mean monoglycerides, diglycerides and the technical mixtures thereof.

With regard to cosmetic action, advantageous cosmetic agents have proven to be those in which the proportion by weight of the oil body in the total weight of the agent amounts to from about 0.01 to about 5.0 wt. %, such as from about 0.02 to about 4.0 wt. %, for example from about 0.05 to about 2.0 wt. %.

The following tables show the composition of some exemplary cosmetic agents (details in wt. % relative to the total weight of the cosmetic agent unless otherwise stated).

| | Formula | Formula | Formula | Formula | Formula |
|---|---|---|---|---|---|
| | 1 | 2 | 3 | 4 | 5 |
| Copolymer A | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Water, misc. | ad 100 | ad 100 | ad 100 | ad 100 | ad 100 |

| | Formula | Formula | Formula | Formula | Formula |
|---|---|---|---|---|---|
| | 6 | 7 | 8 | 9 | 10 |
| Copolymer A¹⁾ | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Water, misc. | ad 100 | ad 100 | ad 100 | ad 100 | ad 100 |

| | Formula | Formula | Formula | Formula | Formula |
|---|---|---|---|---|---|
| | 11 | 12 | 13 | 14 | 15 |
| Copolymer A | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Water, misc. | ad 100 | ad 100 | ad 100 | ad 100 | ad 100 |

| | Formula | Formula | Formula | Formula | Formula |
|---|---|---|---|---|---|
| | 16 | 17 | 18 | 19 | 20 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Water, misc. | ad 100 | ad 100 | ad 100 | ad 100 | ad 100 |

| | Formula | Formula | Formula | Formula | Formula |
|---|---|---|---|---|---|
| | 21 | 22 | 23 | 24 | 25 |
| Copolymer A | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Alkanolamine³⁾ | 80 to 100 | 90 to 100 | 95 to 100 | 98 | 98 |
| Water, misc. | ad 100 | ad 100 | ad 100 | ad 100 | ad 100 |

| | Formula | Formula | Formula | Formula | Formula |
|---|---|---|---|---|---|
| | 26 | 27 | 28 | 29 | 30 |
| Copolymer A¹⁾ | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Alkanolamine³⁾ | 80 to 100 | 90 to 100 | 95 to 100 | 98 | 98 |
| Water, misc. | ad 100 | ad 100 | ad 100 | ad 100 | ad 100 |

| | Formula | Formula | Formula | Formula | Formula |
|---|---|---|---|---|---|
| | 31 | 32 | 33 | 34 | 35 |
| Copolymer A | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Alkanolamine³⁾ | 80 to 100 | 90 to 100 | 95 to 100 | 98 | 98 |
| Water, misc. | ad 100 | ad 100 | ad 100 | ad 100 | ad 100 |

| | Formula | Formula | Formula | Formula | Formula |
|---|---|---|---|---|---|
| | 36 | 37 | 38 | 39 | 40 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Alkanolamine³⁾ | 80 to 100 | 90 to 100 | 95 to 100 | 98 | 98 |
| Water, misc. | ad 100 | ad 100 | ad 100 | ad 100 | ad 100 |

| | Formula | Formula | Formula | Formula | Formula |
|---|---|---|---|---|---|
| | 41 | 42 | 43 | 44 | 45 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| 2-Amino-2- | 80 to 100 | 90 to 100 | 95 to 100 | 98 | 98 |
| methylpro- | | | | | |
| panol³⁾ | | | | | |
| Water, misc. | ad 100 | ad 100 | ad 100 | ad 100 | ad 100 |

| | Formula | Formula | Formula | Formula | Formula |
|---|---|---|---|---|---|
| | 46 | 47 | 48 | 49 | 50 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Alkyltrimeth- | 0.05 to 3.0 | 0.1 to 2.0 | 0.2 to 1.0 | 0.5 | 0.3 |
| ylammonium | | | | | |
| chloride | | | | | |
| Water, misc. | ad 100 | ad 100 | ad 100 | ad 100 | ad 100 |

| | Formula | Formula | Formula | Formula | Formula |
|---|---|---|---|---|---|
| | 51 | 52 | 53 | 54 | 55 |
| Copoly- | 0.05 to 10 | 0.1 to 7.0 | 0.2 to 5.0 | 0.4 | 1.2 |
| mer A¹⁾ | | | | | |
| Copoly- | 0.05 to 10 | 0.1 to 7.0 | 0.2 to 5.0 | 0.8 | 0.8 |
| mer B²⁾ | | | | | |
| Oil bodies | 0.01 to 5.0 | 0.02 to 4.0 | 0.05 to 2.0 | 0.1 | 0.1 |
| Water, | ad 100 | ad 100 | ad 100 | ad 100 | ad 100 |
| misc. | | | | | |

| | Formula | Formula | Formula | Formula | Formula |
|---|---|---|---|---|---|
| | 56 | 57 | 58 | 59 | 60 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Water | 40 to 99 | 50 to 98 | 60 to 95 | 97 | 92 |
| Misc. | ad 100 | ad 100 | ad 100 | ad 100 | ad 100 |

| | Formula | Formula | Formula | Formula | Formula |
|---|---|---|---|---|---|
| | 61 | 62 | 63 | 64 | 65 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Alkyltrimeth- | 0.05 to 3.0 | 0.1 to 2.0 | 0.2 to 1.0 | 0.5 | 0.3 |
| ylammonium | | | | | |
| chloride | | | | | |
| Water | 40 to 99 | 50 to 98 | 60 to 95 | 97 | 92 |
| Misc. | ad 100 | ad 100 | ad 100 | ad 100 | ad 100 |

| | Formula | Formula | Formula | Formula | Formula |
|---|---|---|---|---|---|
| | 66 | 67 | 68 | 69 | 70 |
| Copoly- | 0.05 to 10 | 0.1 to 7.0 | 0.2 to 5.0 | 0.4 | 1.2 |
| mer A¹⁾ | | | | | |
| Copoly- | 0.05 to 10 | 0.1 to 7.0 | 0.2 to 5.0 | 0.8 | 0.8 |
| mer B²⁾ | | | | | |
| Oil bodies | 0.01 to 5.0 | 0.02 to 4.0 | 0.05 to 2.0 | 0.1 | 0.1 |
| Water | 40 to 99 | 50 to 98 | 60 to 95 | 97 | 92 |
| Misc. | ad 100 | ad 100 | ad 100 | ad 100 | ad 100 |

| | Formula | Formula | Formula | Formula | Formula |
|---|---|---|---|---|---|
| | 71 | 72 | 73 | 74 | 75 |
| Copoly- | 0.05 to 10 | 0.1 to 7.0 | 0.2 to 5.0 | 0.4 | 1.2 |
| mer A¹⁾ | | | | | |
| Copoly- | 0.05 to 10 | 0.1 to 7.0 | 0.2 to 5.0 | 0.8 | 0.8 |
| mer B²⁾ | | | | | |
| 2-Amino- | 80 to 100 | 90 to 100 | 95 to 100 | 98 | 98 |
| 2-meth- | | | | | |
| ylpro- | | | | | |
| panol³⁾ | | | | | |
| Oil bodies | 0.01 to 5.0 | 0.02 to 4.0 | 0.05 to 2.0 | 0.1 | 0.1 |
| Water | 40 to 99 | 50 to 98 | 60 to 95 | 97 | 92 |
| Misc. | ad 100 | ad 100 | ad 100 | ad 100 | ad 100 |

¹⁾Polystyrenesulfonate with a molecular weight of 80 to 180 kDa ²⁾Copolymer B formed from 3-methyl-1-vinylimidazolium methylsulfate (B1), N-vinylpyrrolidone (B2), N-vinylimidazole (B3) and methacrylic acid amide (B4), wherein copolymer B consists in a proportion of at least about 95 wt. % of the structural units (B1), (B2), (B3) and (B4).
³⁾% of the quantity required for complete neutralization of copolymer A

Exemplary cosmetic agents may be formulated in any forms conventional for cosmetic agents, for example in the form of solutions, which may for example be applied onto the hair as a hair lotion, in the form of creams, emulsions, waxes, gels or also surfactant-containing foaming solutions or other preparations which are suitable for use on the hair. In an alternative embodiment, these agents may however also assume gel or cream form, wherein transparent gels are more preferred.

Exemplary compositions are in particular highly suitable for stabilizing gas bubbles in the agent. In this way, air or other gases or gas mixtures can be readily incorporated into the agents according to the invention in such a manner as to be stable in the long-term. This may optionally proceed during production of the agents, by exposing the agent to gas, preferably air, before packaging and packaging a product containing visible gas bubbles. The agents according to the invention preferably assume the form of a foam. A foam is here a preparation comprising gas filled bubbles surrounded by liquid (liquid foam) or solid (stiff foam) walls. The compositions listed in the following table are preferably stiff foams. The density of preferred compositions amounts to from about 0.3 to about 1.0 g/cm³, such as from about 0.4 to about 0.9 g/cm³, for example from about 0.5 to about 0.8 g/cm³. Such foams may for example be produced by beating the preparation in a suitable mixer or by exposure to a suitable gas, preferably air.

The following tables show the composition of some exemplary cosmetic foams. In this table the left-hand column (“formula x”) refers in each case to one of the exemplary cosmetic compositions listed in the tables disclosed further above. The further columns two to seven (“density”) in each case indicate the density of the corresponding cosmetic composition.

In other words, a cosmetic preparation according to line 12, column 5 of the following table comprises a cosmetic agent according to formula II with a density of 0.44 g/cm³.

| | Density [g/cm³] | | | | | |
|---|---|---|---|---|---|---|
| Formula 1 | 0.3 to 1.0 | 0.93 | 0.4 to 0.9 | 0.44 | 0.5 to 0.8 | 0.61 |
| Formula 2 | 0.3 to 1.0 | 0.93 | 0.4 to 0.9 | 0.44 | 0.5 to 0.8 | 0.61 |
| Formula 3 | 0.3 to 1.0 | 0.93 | 0.4 to 0.9 | 0.44 | 0.5 to 0.8 | 0.61 |
| Formula 4 | 0.3 to 1.0 | 0.93 | 0.4 to 0.9 | 0.44 | 0.5 to 0.8 | 0.61 |
| Formula 5 | 0.3 to 1.0 | 0.93 | 0.4 to 0.9 | 0.44 | 0.5 to 0.8 | 0.61 |
| Formula 6 | 0.3 to 1.0 | 0.93 | 0.4 to 0.9 | 0.44 | 0.5 to 0.8 | 0.61 |
| Formula 7 | 0.3 to 1.0 | 0.93 | 0.4 to 0.9 | 0.44 | 0.5 to 0.8 | 0.61 |
| Formula 8 | 0.3 to 1.0 | 0.93 | 0.4 to 0.9 | 0.44 | 0.5 to 0.8 | 0.61 |
| Formula 9 | 0.3 to 1.0 | 0.93 | 0.4 to 0.9 | 0.44 | 0.5 to 0.8 | 0.61 |
| Formula 10 | 0.3 to 1.0 | 0.93 | 0.4 to 0.9 | 0.44 | 0.5 to 0.8 | 0.61 |
| Formula 11 | 0.3 to 1.0 | 0.93 | 0.4 to 0.9 | 0.44 | 0.5 to 0.8 | 0.61 |
| Formula 12 | 0.3 to 1.0 | 0.93 | 0.4 to 0.9 | 0.44 | 0.5 to 0.8 | 0.61 |
| Formula 13 | 0.3 to 1.0 | 0.93 | 0.4 to 0.9 | 0.44 | 0.5 to 0.8 | 0.61 |
| Formula 14 | 0.3 to 1.0 | 0.93 | 0.4 to 0.9 | 0.44 | 0.5 to 0.8 | 0.61 |
| Formula 15 | 0.3 to 1.0 | 0.93 | 0.4 to 0.9 | 0.44 | 0.5 to 0.8 | 0.61 |
| Formula 16 | 0.3 to 1.0 | 0.93 | 0.4 to 0.9 | 0.44 | 0.5 to 0.8 | 0.61 |
| Formula 17 | 0.3 to 1.0 | 0.93 | 0.4 to 0.9 | 0.44 | 0.5 to 0.8 | 0.61 |
| Formula 18 | 0.3 to 1.0 | 0.93 | 0.4 to 0.9 | 0.44 | 0.5 to 0.8 | 0.61 |
| Formula 19 | 0.3 to 1.0 | 0.93 | 0.4 to 0.9 | 0.44 | 0.5 to 0.8 | 0.61 |
| Formula 20 | 0.3 to 1.0 | 0.93 | 0.4 to 0.9 | 0.44 | 0.5 to 0.8 | 0.61 |
| Formula 21 | 0.3 to 1.0 | 0.93 | 0.4 to 0.9 | 0.44 | 0.5 to 0.8 | 0.61 |
| Formula 22 | 0.3 to 1.0 | 0.93 | 0.4 to 0.9 | 0.44 | 0.5 to 0.8 | 0.61 |
| Formula 23 | 0.3 to 1.0 | 0.93 | 0.4 to 0.9 | 0.44 | 0.5 to 0.8 | 0.61 |
| Formula 24 | 0.3 to 1.0 | 0.93 | 0.4 to 0.9 | 0.44 | 0.5 to 0.8 | 0.61 |
| Formula 25 | 0.3 to 1.0 | 0.93 | 0.4 to 0.9 | 0.44 | 0.5 to 0.8 | 0.61 |
| Formula 26 | 0.3 to 1.0 | 0.93 | 0.4 to 0.9 | 0.44 | 0.5 to 0.8 | 0.61 |
| Formula 27 | 0.3 to 1.0 | 0.93 | 0.4 to 0.9 | 0.44 | 0.5 to 0.8 | 0.61 |
| Formula 28 | 0.3 to 1.0 | 0.93 | 0.4 to 0.9 | 0.44 | 0.5 to 0.8 | 0.61 |
| Formula 29 | 0.3 to 1.0 | 0.93 | 0.4 to 0.9 | 0.44 | 0.5 to 0.8 | 0.61 |
| Formula 30 | 0.3 to 1.0 | 0.93 | 0.4 to 0.9 | 0.44 | 0.5 to 0.8 | 0.61 |
| Formula 31 | 0.3 to 1.0 | 0.93 | 0.4 to 0.9 | 0.44 | 0.5 to 0.8 | 0.61 |
| Formula 32 | 0.3 to 1.0 | 0.93 | 0.4 to 0.9 | 0.44 | 0.5 to 0.8 | 0.61 |
| Formula 33 | 0.3 to 1.0 | 0.93 | 0.4 to 0.9 | 0.44 | 0.5 to 0.8 | 0.61 |
| Formula 34 | 0.3 to 1.0 | 0.93 | 0.4 to 0.9 | 0.44 | 0.5 to 0.8 | 0.61 |
| Formula 35 | 0.3 to 1.0 | 0.93 | 0.4 to 0.9 | 0.44 | 0.5 to 0.8 | 0.61 |
| Formula 36 | 0.3 to 1.0 | 0.93 | 0.4 to 0.9 | 0.44 | 0.5 to 0.8 | 0.61 |
| Formula 37 | 0.3 to 1.0 | 0.93 | 0.4 to 0.9 | 0.44 | 0.5 to 0.8 | 0.61 |
| Formula 38 | 0.3 to 1.0 | 0.93 | 0.4 to 0.9 | 0.44 | 0.5 to 0.8 | 0.61 |
| Formula 39 | 0.3 to 1.0 | 0.93 | 0.4 to 0.9 | 0.44 | 0.5 to 0.8 | 0.61 |
| Formula 40 | 0.3 to 1.0 | 0.93 | 0.4 to 0.9 | 0.44 | 0.5 to 0.8 | 0.61 |
| Formula 41 | 0.3 to 1.0 | 0.93 | 0.4 to 0.9 | 0.44 | 0.5 to 0.8 | 0.61 |
| Formula 42 | 0.3 to 1.0 | 0.93 | 0.4 to 0.9 | 0.44 | 0.5 to 0.8 | 0.61 |
| Formula 43 | 0.3 to 1.0 | 0.93 | 0.4 to 0.9 | 0.44 | 0.5 to 0.8 | 0.61 |
| Formula 44 | 0.3 to 1.0 | 0.93 | 0.4 to 0.9 | 0.44 | 0.5 to 0.8 | 0.61 |
| Formula 45 | 0.3 to 1.0 | 0.93 | 0.4 to 0.9 | 0.44 | 0.5 to 0.8 | 0.61 |
| Formula 46 | 0.3 to 1.0 | 0.93 | 0.4 to 0.9 | 0.44 | 0.5 to 0.8 | 0.61 |
| Formula 47 | 0.3 to 1.0 | 0.93 | 0.4 to 0.9 | 0.44 | 0.5 to 0.8 | 0.61 |
| Formula 48 | 0.3 to 1.0 | 0.93 | 0.4 to 0.9 | 0.44 | 0.5 to 0.8 | 0.61 |
| Formula 49 | 0.3 to 1.0 | 0.93 | 0.4 to 0.9 | 0.44 | 0.5 to 0.8 | 0.61 |
| Formula 50 | 0.3 to 1.0 | 0.93 | 0.4 to 0.9 | 0.44 | 0.5 to 0.8 | 0.61 |
| Formula 51 | 0.3 to 1.0 | 0.93 | 0.4 to 0.9 | 0.44 | 0.5 to 0.8 | 0.61 |
| Formula 52 | 0.3 to 1.0 | 0.93 | 0.4 to 0.9 | 0.44 | 0.5 to 0.8 | 0.61 |
| Formula 53 | 0.3 to 1.0 | 0.93 | 0.4 to 0.9 | 0.44 | 0.5 to 0.8 | 0.61 |
| Formula 54 | 0.3 to 1.0 | 0.93 | 0.4 to 0.9 | 0.44 | 0.5 to 0.8 | 0.61 |
| Formula 55 | 0.3 to 1.0 | 0.93 | 0.4 to 0.9 | 0.44 | 0.5 to 0.8 | 0.61 |
| Formula 56 | 0.3 to 1.0 | 0.93 | 0.4 to 0.9 | 0.44 | 0.5 to 0.8 | 0.61 |
| Formula 57 | 0.3 to 1.0 | 0.93 | 0.4 to 0.9 | 0.44 | 0.5 to 0.8 | 0.61 |
| Formula 58 | 0.3 to 1.0 | 0.93 | 0.4 to 0.9 | 0.44 | 0.5 to 0.8 | 0.61 |
| Formula 59 | 0.3 to 1.0 | 0.93 | 0.4 to 0.9 | 0.44 | 0.5 to 0.8 | 0.61 |
| Formula 60 | 0.3 to 1.0 | 0.93 | 0.4 to 0.9 | 0.44 | 0.5 to 0.8 | 0.61 |
| Formula 61 | 0.3 to 1.0 | 0.93 | 0.4 to 0.9 | 0.44 | 0.5 to 0.8 | 0.61 |
| Formula 62 | 0.3 to 1.0 | 0.93 | 0.4 to 0.9 | 0.44 | 0.5 to 0.8 | 0.61 |
| Formula 63 | 0.3 to 1.0 | 0.93 | 0.4 to 0.9 | 0.44 | 0.5 to 0.8 | 0.61 |
| Formula 64 | 0.3 to 1.0 | 0.93 | 0.4 to 0.9 | 0.44 | 0.5 to 0.8 | 0.61 |
| Formula 65 | 0.3 to 1.0 | 0.93 | 0.4 to 0.9 | 0.44 | 0.5 to 0.8 | 0.61 |
| Formula 66 | 0.3 to 1.0 | 0.93 | 0.4 to 0.9 | 0.44 | 0.5 to 0.8 | 0.61 |
| Formula 67 | 0.3 to 1.0 | 0.93 | 0.4 to 0.9 | 0.44 | 0.5 to 0.8 | 0.61 |
| Formula 68 | 0.3 to 1.0 | 0.93 | 0.4 to 0.9 | 0.44 | 0.5 to 0.8 | 0.61 |
| Formula 69 | 0.3 to 1.0 | 0.93 | 0.4 to 0.9 | 0.44 | 0.5 to 0.8 | 0.61 |
| Formula 70 | 0.3 to 1.0 | 0.93 | 0.4 to 0.9 | 0.44 | 0.5 to 0.8 | 0.61 |
| Formula 71 | 0.3 to 1.0 | 0.93 | 0.4 to 0.9 | 0.44 | 0.5 to 0.8 | 0.61 |
| Formula 72 | 0.3 to 1.0 | 0.93 | 0.4 to 0.9 | 0.44 | 0.5 to 0.8 | 0.61 |
| Formula 73 | 0.3 to 1.0 | 0.93 | 0.4 to 0.9 | 0.44 | 0.5 to 0.8 | 0.61 |
| Formula 74 | 0.3 to 1.0 | 0.93 | 0.4 to 0.9 | 0.44 | 0.5 to 0.8 | 0.61 |
| Formula 75 | 0.3 to 1.0 | 0.93 | 0.4 to 0.9 | 0.44 | 0.5 to 0.8 | 0.61 |

In an alternative embodiment, exemplary cosmetic agents may be packaged as pump or aerosol sprays. In addition to further active and auxiliary substances, preferred aerosol sprays contain a propellant. Suitable propellants (propellant gases) are propane, n-butane, iso-butane, dimethyl ether (DME), nitrogen, air, nitrous oxide, 1,1-difluoroethane, specifically both individually and in combination. Hydrophilic propellant gases, such as for example carbon dioxide, may also advantageously be used if a small proportion of hydrophilic gases is selected and a lipophilic propellant gas (for example propane/butane) is present in excess. Dimethyl ether, propane, n-butane, iso-butane and mixtures of these propellant gases may be used. An exemplary embodiment uses propane/butane mixtures or isobutane. Exemplary cosmetic agents, relative to the total weight thereof, contain the propellant in a quantity of from about 2.0 to about 20 wt. %, such as from about 4.0 to about 15 wt. %, for example from about 5.0 to about 10 wt. %.

The following tables show the composition of some exemplary propellant-containing cosmetic agents. In this table the left-hand column (“formula x”) refers in each case to one of the exemplary cosmetic compositions listed in the tables disclosed further above. The further columns two to seven (“propellant”) in each case indicate the quantity of propellant added to the corresponding cosmetic composition. These indications in “wt. %” relate to the total weight of the cosmetic composition of the respective “formula x” without propellant.

In other words, a cosmetic preparation according to line 12, column 5 of the following table comprises a 20:1 mixture of the propellant-free cosmetic agent according to formula II with a propane/butane mixture.

| | Propellant [wt. %] | | | | | |
|---|---|---|---|---|---|---|
| Formula 1 | 2.0 to 10 | 5.0 | 2.0 to 10 P/B* | 5.0 P/B | 2.0 to 10 iB** | 5.0 iB |
| Formula 2 | 2.0 to 10 | 5.0 | 2.0 to 10 P/B* | 5.0 P/B | 2.0 to 10 iB** | 5.0 iB |
| Formula 3 | 2.0 to 10 | 5.0 | 2.0 to 10 P/B* | 5.0 P/B | 2.0 to 10 iB** | 5.0 iB |
| Formula 4 | 2.0 to 10 | 5.0 | 2.0 to 10 P/B* | 5.0 P/B | 2.0 to 10 iB** | 5.0 iB |
| Formula 5 | 2.0 to 10 | 5.0 | 2.0 to 10 P/B* | 5.0 P/B | 2.0 to 10 iB** | 5.0 iB |
| Formula 6 | 2.0 to 10 | 5.0 | 2.0 to 10 P/B* | 5.0 P/B | 2.0 to 10 iB** | 5.0 iB |
| Formula 7 | 2.0 to 10 | 5.0 | 2.0 to 10 P/B* | 5.0 P/B | 2.0 to 10 iB** | 5.0 iB |
| Formula 8 | 2.0 to 10 | 5.0 | 2.0 to 10 P/B* | 5.0 P/B | 2.0 to 10 iB** | 5.0 iB |
| Formula 9 | 2.0 to 10 | 5.0 | 2.0 to 10 P/B* | 5.0 P/B | 2.0 to 10 iB** | 5.0 iB |
| Formula 10 | 2.0 to 10 | 5.0 | 2.0 to 10 P/B* | 5.0 P/B | 2.0 to 10 iB** | 5.0 iB |
| Formula 11 | 2.0 to 10 | 5.0 | 2.0 to 10 P/B* | 5.0 P/B | 2.0 to 10 iB** | 5.0 iB |
| Formula 12 | 2.0 to 10 | 5.0 | 2.0 to 10 P/B* | 5.0 P/B | 2.0 to 10 iB** | 5.0 iB |
| Formula 13 | 2.0 to 10 | 5.0 | 2.0 to 10 P/B* | 5.0 P/B | 2.0 to 10 iB** | 5.0 iB |
| Formula 14 | 2.0 to 10 | 5.0 | 2.0 to 10 P/B* | 5.0 P/B | 2.0 to 10 iB** | 5.0 iB |
| Formula 15 | 2.0 to 10 | 5.0 | 2.0 to 10 P/B* | 5.0 P/B | 2.0 to 10 iB** | 5.0 iB |
| Formula 16 | 2.0 to 10 | 5.0 | 2.0 to 10 P/B* | 5.0 P/B | 2.0 to 10 iB** | 5.0 iB |
| Formula 17 | 2.0 to 10 | 5.0 | 2.0 to 10 P/B* | 5.0 P/B | 2.0 to 10 iB** | 5.0 iB |
| Formula 18 | 2.0 to 10 | 5.0 | 2.0 to 10 P/B* | 5.0 P/B | 2.0 to 10 iB** | 5.0 iB |
| Formula 19 | 2.0 to 10 | 5.0 | 2.0 to 10 P/B* | 5.0 P/B | 2.0 to 10 iB** | 5.0 iB |
| Formula 20 | 2.0 to 10 | 5.0 | 2.0 to 10 P/B* | 5.0 P/B | 2.0 to 10 iB** | 5.0 iB |
| Formula 21 | 2.0 to 10 | 5.0 | 2.0 to 10 P/B* | 5.0 P/B | 2.0 to 10 iB** | 5.0 iB |
| Formula 22 | 2.0 to 10 | 5.0 | 2.0 to 10 P/B* | 5.0 P/B | 2.0 to 10 iB** | 5.0 iB |
| Formula 23 | 2.0 to 10 | 5.0 | 2.0 to 10 P/B* | 5.0 P/B | 2.0 to 10 iB** | 5.0 iB |
| Formula 24 | 2.0 to 10 | 5.0 | 2.0 to 10 P/B* | 5.0 P/B | 2.0 to 10 iB** | 5.0 iB |
| Formula 25 | 2.0 to 10 | 5.0 | 2.0 to 10 P/B* | 5.0 P/B | 2.0 to 10 iB** | 5.0 iB |
| Formula 26 | 2.0 to 10 | 5.0 | 2.0 to 10 P/B* | 5.0 P/B | 2.0 to 10 iB** | 5.0 iB |
| Formula 27 | 2.0 to 10 | 5.0 | 2.0 to 10 P/B* | 5.0 P/B | 2.0 to 10 iB** | 5.0 iB |
| Formula 28 | 2.0 to 10 | 5.0 | 2.0 to 10 P/B* | 5.0 P/B | 2.0 to 10 iB** | 5.0 iB |
| Formula 29 | 2.0 to 10 | 5.0 | 2.0 to 10 P/B* | 5.0 P/B | 2.0 to 10 iB** | 5.0 iB |
| Formula 30 | 2.0 to 10 | 5.0 | 2.0 to 10 P/B* | 5.0 P/B | 2.0 to 10 iB** | 5.0 iB |
| Formula 31 | 2.0 to 10 | 5.0 | 2.0 to 10 P/B* | 5.0 P/B | 2.0 to 10 iB** | 5.0 iB |
| Formula 32 | 2.0 to 10 | 5.0 | 2.0 to 10 P/B* | 5.0 P/B | 2.0 to 10 iB** | 5.0 iB |
| Formula 33 | 2.0 to 10 | 5.0 | 2.0 to 10 P/B* | 5.0 P/B | 2.0 to 10 iB** | 5.0 iB |
| Formula 34 | 2.0 to 10 | 5.0 | 2.0 to 10 P/B* | 5.0 P/B | 2.0 to 10 iB** | 5.0 iB |
| Formula 35 | 2.0 to 10 | 5.0 | 2.0 to 10 P/B* | 5.0 P/B | 2.0 to 10 iB** | 5.0 iB |
| Formula 36 | 2.0 to 10 | 5.0 | 2.0 to 10 P/B* | 5.0 P/B | 2.0 to 10 iB** | 5.0 iB |
| Formula 37 | 2.0 to 10 | 5.0 | 2.0 to 10 P/B* | 5.0 P/B | 2.0 to 10 iB** | 5.0 iB |
| Formula 38 | 2.0 to 10 | 5.0 | 2.0 to 10 P/B* | 5.0 P/B | 2.0 to 10 iB** | 5.0 iB |
| Formula 39 | 2.0 to 10 | 5.0 | 2.0 to 10 P/B* | 5.0 P/B | 2.0 to 10 iB** | 5.0 iB |
| Formula 40 | 2.0 to 10 | 5.0 | 2.0 to 10 P/B* | 5.0 P/B | 2.0 to 10 iB** | 5.0 iB |
| Formula 41 | 2.0 to 10 | 5.0 | 2.0 to 10 P/B* | 5.0 P/B | 2.0 to 10 iB** | 5.0 iB |
| Formula 42 | 2.0 to 10 | 5.0 | 2.0 to 10 P/B* | 5.0 P/B | 2.0 to 10 iB** | 5.0 iB |
| Formula 43 | 2.0 to 10 | 5.0 | 2.0 to 10 P/B* | 5.0 P/B | 2.0 to 10 iB** | 5.0 iB |
| Formula 44 | 2.0 to 10 | 5.0 | 2.0 to 10 P/B* | 5.0 P/B | 2.0 to 10 iB** | 5.0 iB |
| Formula 45 | 2.0 to 10 | 5.0 | 2.0 to 10 P/B* | 5.0 P/B | 2.0 to 10 iB** | 5.0 iB |
| Formula 46 | 2.0 to 10 | 5.0 | 2.0 to 10 P/B* | 5.0 P/B | 2.0 to 10 iB** | 5.0 iB |
| Formula 47 | 2.0 to 10 | 5.0 | 2.0 to 10 P/B* | 5.0 P/B | 2.0 to 10 iB** | 5.0 iB |
| Formula 48 | 2.0 to 10 | 5.0 | 2.0 to 10 P/B* | 5.0 P/B | 2.0 to 10 iB** | 5.0 iB |
| Formula 49 | 2.0 to 10 | 5.0 | 2.0 to 10 P/B* | 5.0 P/B | 2.0 to 10 iB** | 5.0 iB |
| Formula 50 | 2.0 to 10 | 5.0 | 2.0 to 10 P/B* | 5.0 P/B | 2.0 to 10 iB** | 5.0 iB |
| Formula 51 | 2.0 to 10 | 5.0 | 2.0 to 10 P/B* | 5.0 P/B | 2.0 to 10 iB** | 5.0 iB |
| Formula 52 | 2.0 to 10 | 5.0 | 2.0 to 10 P/B* | 5.0 P/B | 2.0 to 10 iB** | 5.0 iB |
| Formula 53 | 2.0 to 10 | 5.0 | 2.0 to 10 P/B* | 5.0 P/B | 2.0 to 10 iB** | 5.0 iB |
| Formula 54 | 2.0 to 10 | 5.0 | 2.0 to 10 P/B* | 5.0 P/B | 2.0 to 10 iB** | 5.0 iB |
| Formula 55 | 2.0 to 10 | 5.0 | 2.0 to 10 P/B* | 5.0 P/B | 2.0 to 10 iB** | 5.0 iB |
| Formula 56 | 2.0 to 10 | 5.0 | 2.0 to 10 P/B* | 5.0 P/B | 2.0 to 10 iB** | 5.0 iB |
| Formula 57 | 2.0 to 10 | 5.0 | 2.0 to 10 P/B* | 5.0 P/B | 2.0 to 10 iB** | 5.0 iB |
| Formula 58 | 2.0 to 10 | 5.0 | 2.0 to 10 P/B* | 5.0 P/B | 2.0 to 10 iB** | 5.0 iB |
| Formula 59 | 2.0 to 10 | 5.0 | 2.0 to 10 P/B* | 5.0 P/B | 2.0 to 10 iB** | 5.0 iB |
| Formula 60 | 2.0 to 10 | 5.0 | 2.0 to 10 P/B* | 5.0 P/B | 2.0 to 10 iB** | 5.0 iB |
| Formula 61 | 2.0 to 10 | 5.0 | 2.0 to 10 P/B* | 5.0 P/B | 2.0 to 10 iB** | 5.0 iB |
| Formula 62 | 2.0 to 10 | 5.0 | 2.0 to 10 P/B* | 5.0 P/B | 2.0 to 10 iB** | 5.0 iB |
| Formula 63 | 2.0 to 10 | 5.0 | 2.0 to 10 P/B* | 5.0 P/B | 2.0 to 10 iB** | 5.0 iB |
| Formula 64 | 2.0 to 10 | 5.0 | 2.0 to 10 P/B* | 5.0 P/B | 2.0 to 10 iB** | 5.0 iB |
| Formula 65 | 2.0 to 10 | 5.0 | 2.0 to 10 P/B* | 5.0 P/B | 2.0 to 10 iB** | 5.0 iB |
| Formula 66 | 2.0 to 10 | 5.0 | 2.0 to 10 P/B* | 5.0 P/B | 2.0 to 10 iB** | 5.0 iB |
| Formula 67 | 2.0 to 10 | 5.0 | 2.0 to 10 P/B* | 5.0 P/B | 2.0 to 10 iB** | 5.0 iB |
| Formula 68 | 2.0 to 10 | 5.0 | 2.0 to 10 P/B* | 5.0 P/B | 2.0 to 10 iB** | 5.0 iB |
| Formula 69 | 2.0 to 10 | 5.0 | 2.0 to 10 P/B* | 5.0 P/B | 2.0 to 10 iB** | 5.0 iB |
| Formula 70 | 2.0 to 10 | 5.0 | 2.0 to 10 P/B* | 5.0 P/B | 2.0 to 10 iB** | 5.0 iB |
| Formula 71 | 2.0 to 10 | 5.0 | 2.0 to 10 P/B* | 5.0 P/B | 2.0 to 10 iB** | 5.0 iB |
| Formula 72 | 2.0 to 10 | 5.0 | 2.0 to 10 P/B* | 5.0 P/B | 2.0 to 10 iB** | 5.0 iB |
| Formula 73 | 2.0 to 10 | 5.0 | 2.0 to 10 P/B* | 5.0 P/B | 2.0 to 10 iB** | 5.0 iB |
| Formula 74 | 2.0 to 10 | 5.0 | 2.0 to 10 P/B* | 5.0 P/B | 2.0 to 10 iB** | 5.0 iB |
| Formula 75 | 2.0 to 10 | 5.0 | 2.0 to 10 P/B* | 5.0 P/B | 2.0 to 10 iB** | 5.0 iB |

*“P/B” corresponds to a propane/butane mixture
**“iB” corresponds to isobutane

As explained above, exemplary agents have advantageous hair-fixing properties. The disclosure provides a method for temporarily deforming keratinic fibers, in which an exemplary composition is applied onto the keratinic fibers. The present disclosure also provides use of a cosmetic agent for temporarily deforming keratinic fibers. As explained above, exemplary agents are distinguished in particular by improved hold in the case of temporary deformation of keratinic fibers. The disclosure additionally provides the use of a cosmetic agent to improve hold in the case of temporary deformation of keratinic fibers.

### Examples

The humidity resistance (High Humidity Curl Retention; HHCR) of the temporary hair deformation achieved using the following three hair cosmetic agents was determined

| | Invention 1 | Comparison 1 | Comparison 2 |
|---|---|---|---|
| Flexan II¹ | 0.75 | 1.25 | — |
| Luviquat Supreme² | 3.75 | — | 6.25 |
| Sodium benzoate | 0.3 | 0.3 | 0.3 |
| D-Panthenol (75%) | 0.2 | 0.2 | 0.2 |
| Dow Corning 939³ | 0.2 | 0.2 | 0.2 |
| Dehyquart A CA⁴ | 1.0 | 1.0 | 1.0 |
| Castor Oil | 0.2 | 0.2 | 0.2 |
| hydrogenated, 40 EO | | | |
| Perfume | 0.1 | 0.1 | 0.1 |
| Water, misc. | ad 100 | ad 100 | ad 100 |

¹Polystyrenesulfonate (INCI: Sodium Polystyrene Sulfonate, 100%)
²N-Methylvinylimidazole/vinylpyrrolidone/vinylimidazole/methacrylamide copolymer (INCI: Polyquaternium-68, 20% in water)
³Silicone preparation (INCI: Amodimethicone, Trideceth-12, Cetrimonium Chloride)
⁴Trimethylhexadecylammonium chloride

To determine High Humidity Curl Retention, standardized strands of hair from Kerling (item no. 827560) of the “European Natural” hair type, color 6/0, of a length (Lₘₐₓ) of 220 mm and a weight of 0.6 g were used. The strands were washed with a 12.5 wt. % sodium laureth sulfate solution by way of preparation. The strands of hair were dried overnight in a drying oven at 318 K.

0.18 g of the compositions were applied onto a strand of hair and rubbed in. The strand was then wound onto a curler (Fripac-medis, diameter 7 mm, item no. D-1203) and dried overnight at room temperature.

The curlers were carefully removed and the strands hung up. The length of the curls were in each case measured (L₀) and the strands placed in a conditioning cabinet. They were stored there at 294 K and a relative atmospheric humidity of 85% over a period of 6 h, after which the length of the curls was remeasured (L₁).

Five test strands per composition were correspondingly treated and measured.

High Humidity Curl Retention (HHCR) was calculated according to the following formula and the arithmetic mean of the HHCR values for the 5 test strands was determined for each composition:

$$H H C R = \frac{{L}_{max} - {L}_{t}}{{L}_{max} - {L}_{0}}$$

| | Invention 1 | Comparison 1 | Comparison 2 |
|---|---|---|---|
| HHCR | 89% | 49% | 61% |

The measurement data reveal the synergistic action of the polymer combination according to the invention in composition Invention 1.

1-10. (canceled)

11. A cosmetic agent, containing in a cosmetically acceptable carrier
a) at least one polystyrenesulfonate A
b) at least one copolymer B formed from
at least one monomer (B1) selected from quaternized N-vinylimidazoles, and
at least one monomer (B2) selected from N-vinyl lactams.

12. The cosmetic agent according to claim 11, characterized in that the polystyrenesulfonate has a molecular weight of from about 80 to about 180 kDa.

13. The cosmetic agent according to claim 11, characterized in that the polystyrenesulfonate has a molecular weight of from about 100 to about 160 kDa.

14. The cosmetic agent according to claim 11, characterized in that the polystyrenesulfonate has a molecular weight of from about 120 to about 140 kDa.

15. The cosmetic agent according to claim 11, characterized in that the proportion by weight of the polystyrenesulfonate A in the total weight of the agent amounts to from about 0.05 to about 10 wt. %.

16. The cosmetic agent according to claim 11, characterized in that the proportion by weight of the polystyrenesulfonate A in the total weight of the agent amounts to from about 0.1 to about 7.0 wt. %.

17. The cosmetic agent according to claim 11, characterized in that the proportion by weight of the polystyrenesulfonate A in the total weight of the agent amounts to from about 0.2 to about 5.0 wt. %.

18. The cosmetic agent according to claim 11, characterized in that copolymer B is formed from
3-methyl-1-vinylimidazolium methylsulfate (B 1),
at least one monomer (B2) selected from N-vinylcaprolactam and N-vinylpyrrolidone,
N-vinylimidazole (B3), and
at least one monomer (B4) selected from acrylic acid amide and methacrylic acid amide.

19. The cosmetic agent according to claim 11, characterized in that the proportion by weight of copolymer B in the total weight of the agent amounts to from about 0.05 to about 10 wt. %.

20. The cosmetic agent according to claim 11, characterized in that the proportion by weight of copolymer B in the total weight of the agent amounts to from about 0.1 to about 7.0 wt. %.

21. The cosmetic agent according to claim 11, characterized in that the proportion by weight of copolymer B in the total weight of the agent amounts to from about 0.2 to about 5.0 wt. %.

22. The cosmetic agent according to claim 11, characterized in that the weight ratio of polymers A and B amounts to from about 8:1 to about 1:8.

23. The cosmetic agent according to claim 11, characterized in that the weight ratio of polymers A and B amounts to from about 6:1 to about 1:6.

24. The cosmetic agent according to claim 11, characterized in that the weight ratio of polymers A and B amounts to from about 4:1 to about 1:4.

25. The cosmetic agent according to claim 11, further comprising at least one monomeric quaternary ammonium compound.

26. The cosmetic agent according to claim 11, further comprising a monomeric quaternary ammonium compound selected from the group of trimethylalkylammonium halides.

27. The cosmetic agent according to claim 11, further comprising at least one oil body.

28. The cosmetic agent according to claim 11, further comprising at least one oil body from the group of silicone oils.

29. A method for temporarily deforming keratinic fibers comprising applying onto the keratinic fibers a cosmetic agent, containing in a cosmetically acceptable carrier
a) at least one polystyrenesulfonate A
b) at least one copolymer B formed from
at least one monomer (B1) selected from quaternized N-vinylimidazoles, and
at least one monomer (B2) selected from N-vinyl lactams.
